(19) United States
(12) Plant Patent Application Publication
(10) Pub. No.: US 2015/0296674 P1
(43) Pub. Date: Oct. 15, 2015

(54) Hydrangea Plant Named 'BCHY-06.014'

(50) ;

(71) Applicant: Harrison M. Higaki, San Mateo, CA (US)

(72) Inventor: Harrison M. Higaki, San Mateo, CA (US)

(21) Appl. No.: 13/999,982

(22) Filed: Apr. 11, 2014

(51) Int. Cl. A01H 5/00

(52) U.S. Cl. PLT/250

(57) ABSTRACT

A new and distinct cultivar of Hydrangea macrophylla (Thunb.) named ‘BCHY-06.014’ originated as a controlled cross between varieties. The cultivar ‘BCHY-06.014’ can be blue or red depending on the acidity of the soil and the presence of aluminum. The variety ‘BCHY-06.014’ has attractive inflorescences with relatively large sepalous florets, distinct sepal pigmentation and good commercial characteristics. When grown in the presence of aluminum, the sepals are R.H.S. 104 A (blue group).

### BOTANICAL CLASSIFICATION

Hydrangea macrophylla (Thunb.) ‘BCHY-06.014’

### VARIETY DENOMINATION

‘BCHY-06.014’

### BACKGROUND OF THE INVENTION

This invention relates to a new and distinct cultivar of the Saxifragaceae family. The botanical name of the plant is Hydrangea macrophylla (Thunb.) ‘BCHY-06.014’.

The new cultivar originated as a seedling from a controlled cross between the unpatented variety known as ‘LK49’ which was the seed parent and a commercial variety known as ‘Venedig’ to the inventor which may be the subject of U.S. Plant Pat. No. 10,928 and registered as ‘Venice Raven’. ‘Venedig’ was the pollen parent. ‘LK49’ is relatively compact plant with wiry stems, relatively small leaves, relatively small sepalous florets, and inflorescences that are resistant to being damaged by conditions in commercial coolers.

The variety ‘BCHY-06.014’ has strong stems, attractive inflorescences, and develops very deep pigmentation when treated with aluminum at commercial levels, and even if treated with only a relatively small amount of aluminum it develops a uniform and attractive pigmentation. The variety ‘BCHY-06.014’ has pigmented sepals, and can be grown in soil conditions treated with aluminum to produce blue pigmentation, as is described here. The color of the sepals changes as the plant ages. Below is a table comparing the new variety to similar varieties.

TABLE 1

| | | | | Commerical |
|---|---|---|---|---|
| | | | | variety |
| | | | | ′Venedig′ |
| | | | | which may |
| | | US Plant | US Plant | be US Plant |
| | New Variety | Patent 23,757 | Patent18,593 | Patent 10,928 |
| | ′BCHY-06.014′ | ′BC6.1′ | ′True Blue′ | ′Venice Raven′ |
| Leaf size | 9 cm wide × | 12 cm wide × | Unknown | 11 cm wide × |
| | 13.5 cm long | 15 cm long | | 15.5 cm long- |
| | | | | source US |
| | | | | PP10,928 |
| Plant | 18″ in 6″ pot. | 15″ in 6″ pot. | Unknown | 12″ in 6″ pot- |
| height | | | | observed |
| | | | | controls |
| | | | | grown |
| | | | | alongside |
| | | | | new variety. |
| Stem | Strong | Stems are | Strong | Strong- |
| strength | | strong but | | observed |
| | | benefit from | | controls |
| | | being staked | | grown |
| | | | | alongside |
| | | | | new variety |
| Sepal | Treated With | Upper side | Both sides | Upper side |
| Pigmen- | Aluminum- | of sepals is | of sepalsa | of sepals is |
| tation | Mature | R.H.S. 86 A | are R.H.S. | R.H.S. 84 A |
| | inflorescences- | (violet group); | 100 D (blue | (violet group). |
| | Both sides of | Under side of | group). | Under side |
| | sepals are | sepals is | | of sepals is |
| | R.H.S. 95 B | R.H.S. 88 D | | R.H.S. 85 A |
| | (violet-blue | (violet group) | | (violet group) |
| | group). | | | observed |
| | | | | controls |
| | | | | grown |
| | | | | alongside |
| | | | | new variety. |
| Sepalous | 60 mm | 60 mm to | 50 mm to | 70 mm- |
| Floret | | 70 mm | 60 mm | observed |
| Size- | | | | controls |
| Diameter | | | | grown |
| | | | | alongside |
| | | | | new variety |

The new cultivar ‘BCHY-06.014’ has been successfully asexually reproduced under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in Half Moon Bay, Calif. show that the combination of characteristics as herein disclosed for ‘BCHY-06.014’ remains firmly fixed.

### DESCRIPTION OF THE DRAWINGS

The accompanying drawings consist of color photographs that show the typical plant form, including the inflorescence, foliage, and sepals. The plant shown in FIG. 2 is a different specimen from the specimen shown in the remaining figures. The amount of aluminum used to cause blue pigmentation in the specimen shown in FIG. 2 may have been different from the specimen shown in the other figures. Also this specimen may have been a different age from the plant shown in the other figures when the photograph was taken. Color determinations were made based on specimens represented by the specimen shown in FIGS. 1, 3, 4, 5 and 6.

FIG. 1 is a view of the entire plant showing its form, growth habit, dark green foliage, inflorescence, and the color of its sepals.

FIG. 2 is a view of infloresences of the plant. As noted above, this is a different specimen grown under different conditions than the specimen shown in FIG. 1.

FIG. 3 is a close-up view of the base of the stem of the specimen shown in FIG. 1.

FIG. 4 is a close-up view of the adaxial surface of a mature leaf of the specimen shown in FIG. 1.

FIG. 5 is close-up view of the underside of a panicle of the new variety.

FIG. 6 is a close-up view of florets of the new variety.

### DESCRIPTION OF THE NEW PLANT

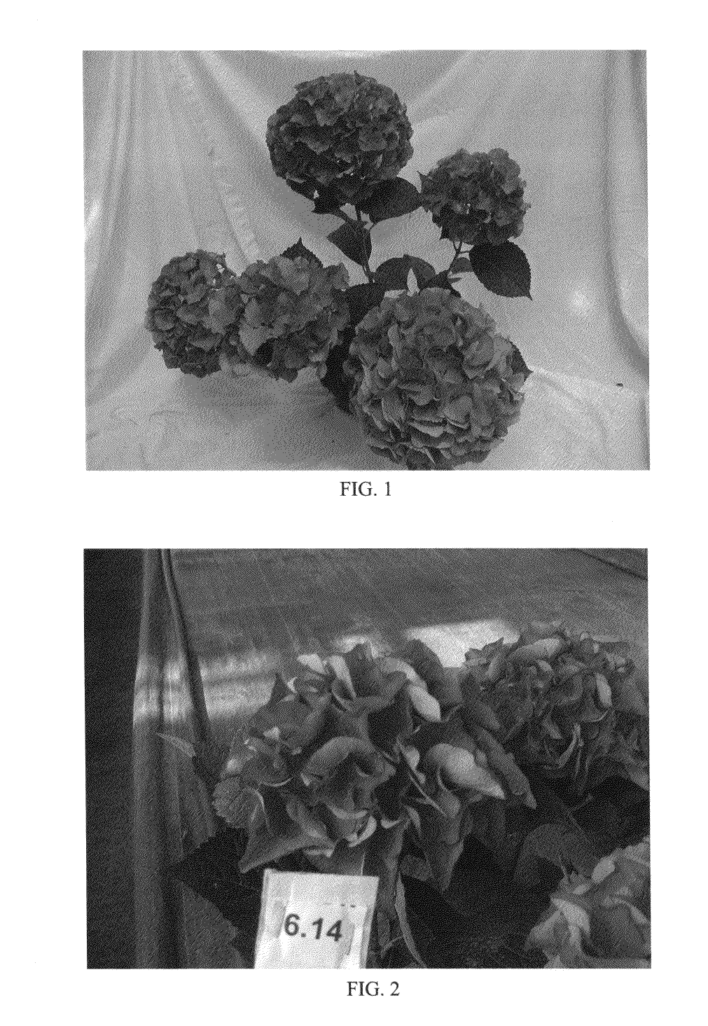

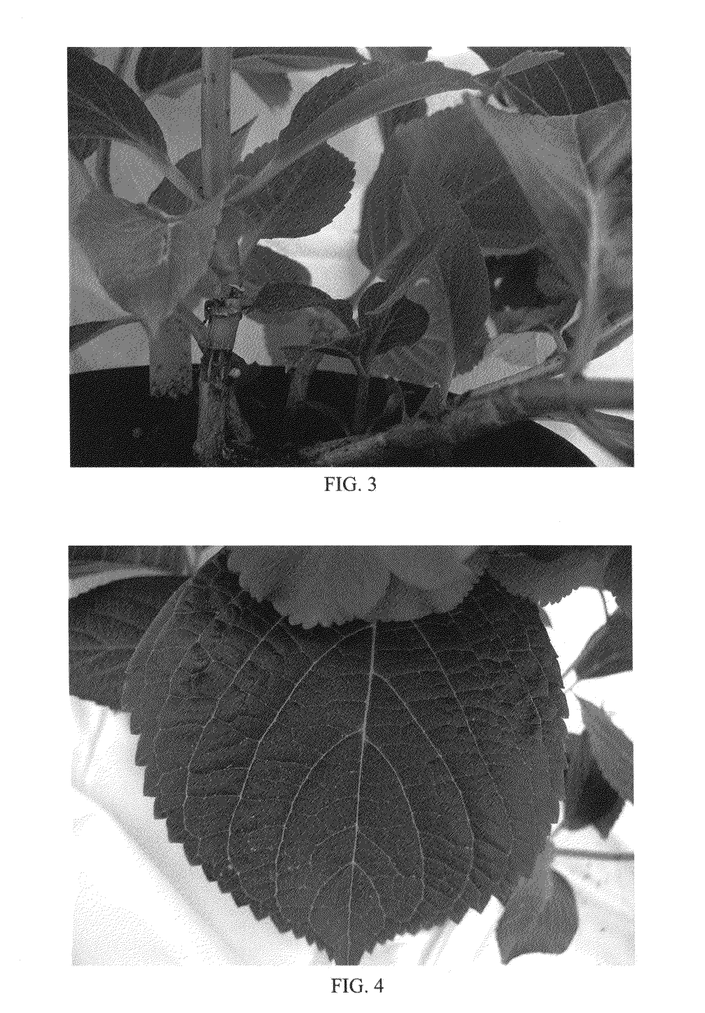

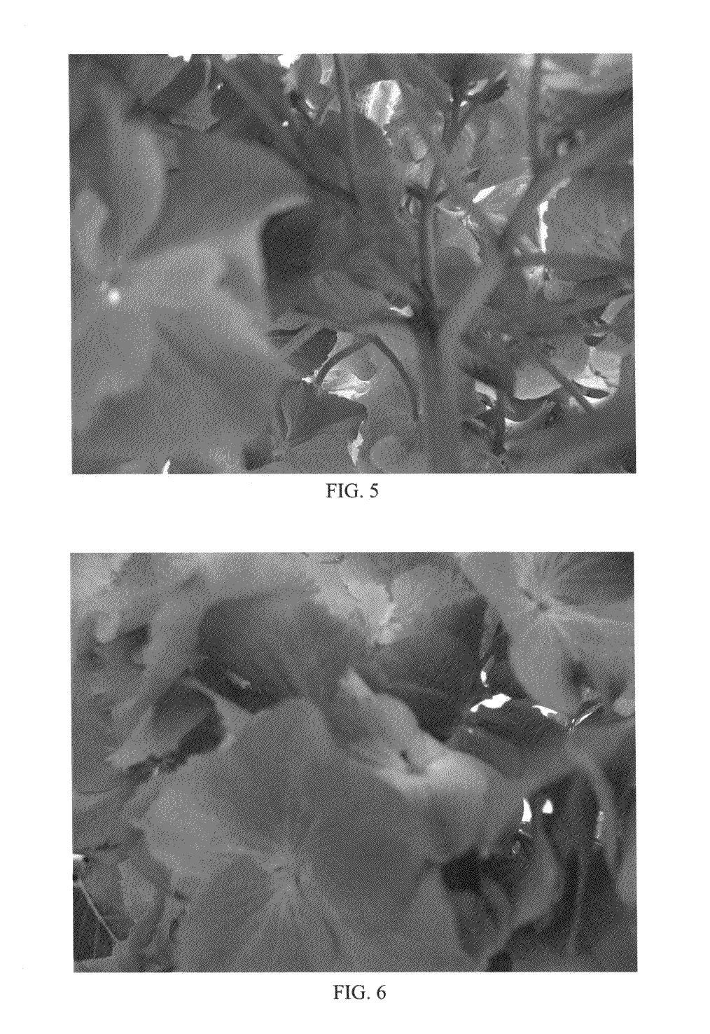

The plant shown in FIGS. 1, 3, 4, 5 and 6 is approximately a year old. The plant started out as cuttings, taken from the stem of a grown plant. The cuttings were placed in a pot and the soil was periodically treated with aluminum to produce blue pigmentation. The plant was pinched early to promote lateral branches.

‘BCHY-06.014’ has not been observed under all possible environmental conditions. The phenotype may vary significantly with variations in environment such as temperature, light intensity, treatment with aluminum and day length. Color determinations were made with The Royal Horticultural Society (R.H.S.) Colour Chart, developed in association with the Flower Council of Holland, located in Lieden.
The plant:
   Origin.—Controlled cross. The new cultivar originated as a seedling from a controlled cross between the unpatented variety known as ‘LK49’ which was the seed parent and the commercial variety known to the inventor as ‘Venedig’ and which may be ‘Venice Raven’, U.S. Plant Pat. No. 10,928, which was the pollen parent.
   Form.—Upright, compact shrub. A typical plant with a mature inflorescence that is ready for sale is approximately 18″ high and has a diameter of 16″ when grown in a 6″ pot with appropriate soil amendments.
   Growth.—Upright, vigorous growth habit. Inflorescence is large. The plant branches easily with shoots forming at the base of the plant. Lateral branches are similar in appearance and form to the main stems.
   Stems.—Lenticels are present. Lenticels are R.H.S. 86 A (violet group) and are 1 to 2 mm long. The surface of young stems is glabrous. Stems become woody as they age. The color of typical young stems and young lateral branches is R.H.S. 144 A (green group). The older portions of the stems are R.H.S. 199 A (grey-brown group). Younger portions of the stems are 5 to 7 mm in diameter. Older portions of the stems are 5 to 7 mm in diameter.
   Foliage.—Abundant. Leaves are opposite on stem and lateral branches. Shape of leaf. — Elliptic with acute base and acute apex. Margins are serrate. Texture. — Glabrous; veins dominate on the underside of the leaf and are sunken on the upper leaf surface. Color. — Mature leaves have an upper side that is R.H.S. 147 A (yellow-green group), and an under side that is R.H.S. 138 B (green group). Leaves are pinnately veined. The midvein and veins branching off the midvein are large and prominent on the underside of the leaves. Veins are R.H.S. 144 D (yellow-green group). Leaves are as wide as 9 cm and 13.5 cm long. Petioles are smooth and approximately 2.5 cm long and 4 mm wide. Petioles are R.H.S. 144 D (yellow-green group).
Buds:
   Form.—Globose with 4 to 5 connate, elliptic, smooth petals. Most buds, whether they will mature into sepalous or non-sepalous florets, have 4 petals. Buds in the center of the inflorescence are non-sepalous. The majority of buds will develop into sepalous florets. They are approximately 1 mm by 1 mm when very young. Buds can be 5 mm in diameter and still unopened. Color of mature buds is R.H.S. 95 B (violet-blue group).
   Aspect.—Smooth.
   Arrangement.—Borne on branched panicles.
Inflorescence:
   Form.—Paniculate. Terminal. As many as 100 individual flowers (florets) per inflorescence. Both sepalous florets and non-sepalous florets borne on the same panicle. Flowers do not produce a fragrance. The peduncle for the inflorescence is strong and upright. Very few non-sepalous florets developing early on cymes that are later hidden by sepalous florets. Florets, both sepalous and non-sepalous, have anthers and style. Inflorescences are long-lasting, up to six weeks.
   Size of inflorescence.—Compact and globose. Individual inflorescence size is dependent on the number of florets. A typical inflorescence can grow as large as 7″ in diameter, and 4.5″ high.
   Shape.—Clusters of numerous small florets; sepalous florets overlap one another. Sepals are persistent.
   Appearance.—Showy.
Florets:
   General.—The non-sepalous florets at the center of the inflorescence open first. Sepalous and non-sepalous florets are perfect and complete. Corolla: Generally, for both sepalous and non-sepalous florets there are 4 petals which fall off as flower matures. Petals are typically 4 mm long and 3 mm wide. Petals are R.H.S. 95 B (violet-blue group). Lenticels are present on pedicels of both sepalous and non-sepalous florets, lenticels are no more than 1 mm to 4mm long and very narrow. Lenticels are R.H.S. 59 B (red-purple group). Pedicel length for non-sepalous florets averages 4 mm. Pedicel length of sepalous florets is approximately 30 mm in length for plants of this age. Pedicels of both sepalous and non-sepalous florets continue to elongate as the inflorescence ages. Pedicels range from 95 A (violet-purple group), to R.H.S. 95 C (violet-purple group).
   Stamens.—8 to 10 stamens. Pollen is 158 (yellow-white group). Filament is approximately 7 mm long. Filament is R.H.S. 95 B (violet-blue group). Anther is 1 mm long and is regular and basally attached.
   Stigma.—Two to three style each, although most florets have two style. Each style has one stigma. Style is typically 1 mm long. Style is R.H.S. 95 B (violet-blue group). Stigma is R.H.S. 95 B (violet-blue group).
   Ovary.—Ovary is partially inferior.
   Sepalous florets.—General. — Veins dominate on the underside of the sepals. Number of sepals. — 3 to 5 sepals per floret, usually 4. Aspect of sepals. — Smooth and glaucescent. Shape of sepals. — Reniform with acuminate apex. Edges are entire, but with much crenation. Size of sepals. — As the florets mature, the sepals enlarge and overlap each other more and more, until, often, there is no space between the sepals when the petals of the florets open. Sepals at maturity are typically 40 mm long and 43 mm wide. Flowers are typically 60 mm in diameter. The uppersides of the sepals are R.H.S. 104 A (blue group) and the undersides are the same, with the color changing to R.H.S. 96 B (violet-blue group) as the plant ages. Pigmentation develops at the tips of the sepals and travels inward towards base of the sepals.
   Fruit.—none.
   Disease and pest resistance.—unknown.

I claim:

1. A new and distinct Hydrangea macrophylla plant named ‘BCHY-06.014’ substantially as herein shown and described.
